(19) United States
(12) Patent Application Publication
(10) Pub. No.: US 2023/0181778 A1
(43) Pub. Date: Jun. 15, 2023

(54) DISINFECTION MODULE

(71) Applicant: ADATA TECHNOLOGY CO., LTD., New Taipei City (TW)

(72) Inventors: Yu-Syuan Tsai, New Taipei City (TW); Kuo-Ting Chung, Taoyuan City (TW); Chien-Ta Tseng, Kaohsiung City (TW); Chia-Hsiang Hsiao, New Taipei City (TW)

(21) Appl. No.: 18/075,361

(22) Filed: Dec. 5, 2022

Related U.S. Application Data
(60) Provisional application No. 63/289,645, filed on Dec. 15, 2021.

(30) Foreign Application Priority Data
Nov. 25, 2022 (TW) 111145317

(51) Int. Cl. A61L 2/10

(52) U.S. Cl. CPC A61L 2/10; A61L 2202/14; A61L 2202/16

(57) ABSTRACT

A disinfection module includes a base, a plurality of first angle adjustment elements, a plurality of first disinfection light sources and a driving element. The first angle adjustment elements are respectively disposed on the base. The first disinfection light sources are respectively connected to the first angle adjustment elements. The driving element is electrically connected to the first angle adjustment elements and is adapted to drive the first angle adjustment elements to rotate relative to the base, so as to change irradiation angles of the first disinfection light sources.

### CROSS-REFERENCE TO RELATED APPLICATIONS

This application claims the benefit of Taiwanese Application No. 111145317, filed on Nov. 25, 2022, and U.S. Provisional Application No. 63/289,645, filed on Dec. 15, 2021, which are hereby incorporated by reference for all purposes as if fully set forth herein.

### FIELD OF THE INVENTION

The present disclosure relates to an illumination module, and more particularly to a disinfection module.

### BACKGROUND OF THE INVENTION

Disinfection has become an important routine for various public places. For example, disinfection will be executed periodically in most environments such as public consuming places, mass transportation, office buildings and the like with large flow of people. In addition, cleaning and disinfection are further concerned in medical places to prevent potential viral cross infection.

Generally speaking, disinfection in prior art is executed by way of manually spraying disinfectants or spraying disinfectants by machines. However, the disinfectants are not suitable for cloth, and chemical compositions of the disinfectants are likely to cause adverse effects on human body. Therefore, a part of disinfection operations will be executed by way of ultraviolet irradiation so as to provide a more complete disinfection effect.

### SUMMARY OF THE INVENTION

The present disclosure provides a disinfection module to have various irradiation angles of a disinfection light source.

The disinfection module provided by the present disclosure includes a base, a plurality of first angle adjustment elements, a plurality of first disinfection light sources and a driving element. The first angle adjustment elements are respectively disposed on the base. The first disinfection light sources are respectively connected to the first angle adjustment elements. The driving element is electrically connected to the first angle adjustment elements, and the driving element is adapted to drive the first angle adjustment elements to rotate relative to the base, so as to change irradiation angles of the first disinfection light sources.

In an embodiment of the present disclosure, each of the first angle adjustment elements may include a first platform and a first pivoting component. The first pivoting component is pivoted to the first platform and the base, and the first disinfection light sources are respectively connected to the first platforms. The driving element is electrically connected to the first pivoting component, and the driving element is adapted to drive the first pivoting component to rotate.

In an embodiment of the present disclosure, the base may have a side surface, a first surface and a second surface. The first surface is opposite to the second surface. The side surface is connected to the first surface, and the first surface is encircled by the side surface. The first surface has a plurality of recessed parts. The recessed parts are sunken towards the second surface and extend to the side surface. The first angle adjustment elements and the first disinfection light sources are disposed in the recessed parts.

In an embodiment of the present disclosure, for example, the disinfection module further includes a lifting base and a plurality of second disinfection light sources. The lifting base includes a base body and a connecting element, wherein the connecting element is connected to the base body and the base. The driving element is further electrically connected to the connecting element and is further adapted to drive the base body to ascend and descend relative to the base via the connecting element. The second disinfection light sources are connected to the base body.

In an embodiment of the present disclosure, the connecting element includes two first rod bodies and two second rod bodies. The two first rod bodies are disposed across each other and the two first rod bodies each has a first pivoting part, a first end and a second end. The first end is opposite to the second end. The first pivoting part is located between the first end and the second end, and the two first pivoting parts are pivoted to each other. The two first ends are connected to the base body, and the two second ends are connected to the base. The two second rod bodies are disposed across each other and the two first rod bodies each has a second pivoting part, a third end and a fourth end. The third end is opposite to the fourth end. The second pivoting part is located between the third end and the fourth end, and the two second pivoting parts are pivoted to each other. The two third ends are connected to the base body, and the two fourth ends are connected to the base. The driving element is further adapted to drive the two second ends to be close to each other and drive the two fourth ends to be close to each other, so that the base body is adapted to ascend relative to the base. The driving element is further adapted to drive the two second ends to be away from each other and drive the two fourth ends to be away from each other, so that the base body is adapted to descend relative to the base.

In an embodiment of the present disclosure, for example, the connecting element includes a screw rod element. The base has a screw hole. The screw rod element is disposed at the screw hole and is connected to the base body. The driving element is adapted to drive the screw rod element to rotate relative to the base at the screw hole, so that the base body is adapted to ascend or descend relative to the base.

In an embodiment of the present disclosure, for example, the disinfection module further includes a plurality of second angle adjustment elements. The second angle adjustment elements are disposed on the base body and the second disinfection light sources are respectively connected to the second angle adjustment elements. The driving element is further electrically connected to the second angle adjustment elements and is further adapted to drive the second angle adjustment elements to rotate relative to the base body, so as to change the irradiation angles of the second disinfection light sources.

In an embodiment of the present disclosure, each of the second angle adjustment elements includes a second platform and a second pivoting component. The second pivoting component is pivoted to the second platform and the base body, and the second disinfection light sources are respectively connected to the second platforms. The driving element is electrically connected to the second pivoting component and is adapted to drive the second pivoting component to rotate.

In an embodiment of the present disclosure, for example, the base has a first surface. The first surface faces the base body, and the connecting element is connected to the first surface and the base body. The first surface has an accommodation groove, and the base body is adapted to be disposed in the accommodation groove.

In an embodiment of the present disclosure, for example, the disinfection module can further include a moving element, wherein the base is disposed on the moving element. The moving element includes a computing component and a moving component. The computing component is electrically connected to the moving component and is adapted to drive the moving component to actuate. The moving element is adapted to be moved by means of actuation of the moving component.

The disinfection module provided by the present disclosure uses the first disinfection light sources and the first angle adjustment elements, wherein the first angle adjustment elements are connected to the first disinfection light sources, and the first angle adjustment elements can be driven by the driving element to change the irradiation angles of the first disinfection light sources. Therefore, the first disinfection light sources of the disinfection module provided by the present disclosure have variable irradiation angles, thereby increasing the irradiation ranges of the distinction light sources.

### BRIEF DESCRIPTION OF THE DRAWINGS

FIG. 1 is a schematic diagram of a disinfection module in an embodiment of the present disclosure.

FIG. 2 is a block diagram of the disinfection module in FIG. 1.

FIG. 3 is an enlarged drawing of a first disinfection light source and a first angle adjustment element in FIG. 1.

FIG. 4 is an actuating diagram of the first disinfection light source and the first angle adjustment element in FIG. 1.

FIG. 5 is a block diagram of a moving element in FIG. 1.

FIG. 6 is an actuating diagram of a second rod body of a lifting base in FIG. 1.

FIG. 7 is an actuating diagram of a second disinfection light source and a second angle adjustment element in FIG. 1.

FIG. 8 is a schematic diagram showing separation of a connecting element of the disinfection module and a base in another embodiment of the present disclosure.

FIG. 9 is a schematic diagram of a disinfection module in another embodiment of the present disclosure.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

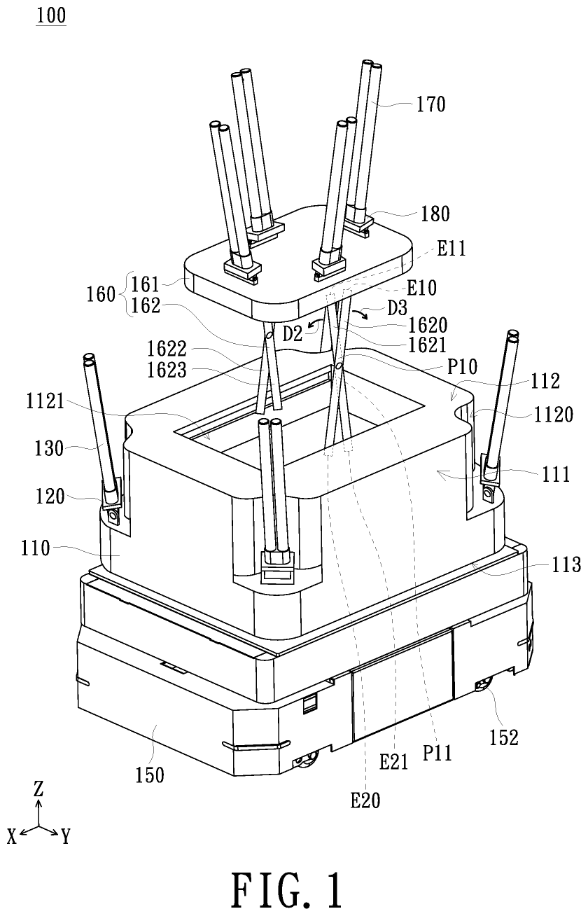

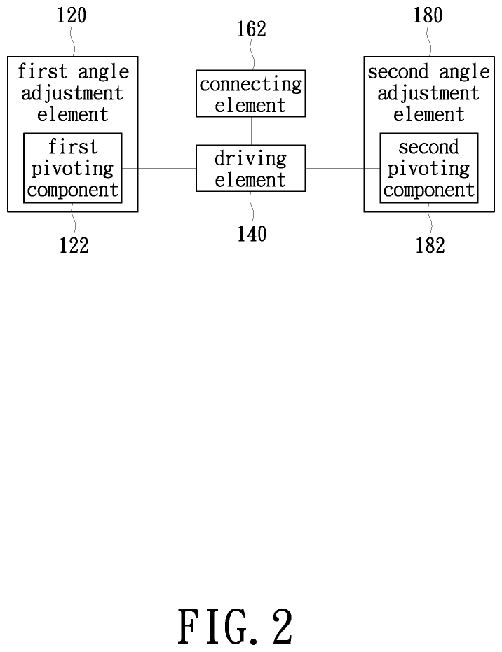

FIG. 1 is a schematic diagram of a disinfection module in an embodiment of the present disclosure. FIG. 2 is a block diagram of the disinfection module in FIG. 1. Please refer to FIG. 1 and FIG. 2, the disinfection module 100 includes a base 110, a plurality of first angle adjustment elements 120, a plurality of first disinfection light sources 130 and a driving element 140 (shown in FIG. 2). The first angle adjustment elements 120 are respectively disposed on the base 110. The first disinfection light sources 130 are respectively connected to the first angle adjustment elements 120. The driving element 140 is electrically connected to the first angle adjustment elements 120, and the driving element 140 is adapted to drive the first angle adjustment elements 120 to rotate relative to the base 110 so as to change irradiation angles of the first disinfection light sources 130.

Please continue to refer to FIG. 1, each of the first distinction light sources 130 includes, for example, an ultraviolet light disinfection light source. Specifically, the ultraviolet light disinfection light source is able to generate Ultra Violet C (UVC) radiation. Further, in this embodiment, the first disinfection light source 130 may include an UVC lamp tube, but the present disclosure is not limited thereto. For example, in an embodiment, the first disinfection light source 130 may include a UVC light emitting diode (UVC LED). It is to be understood that although the first disinfection light source 130 in FIG. 1 is shown in a cylindrical shape, the shape of the first disinfection light source 130 is not limited in the present disclosure.

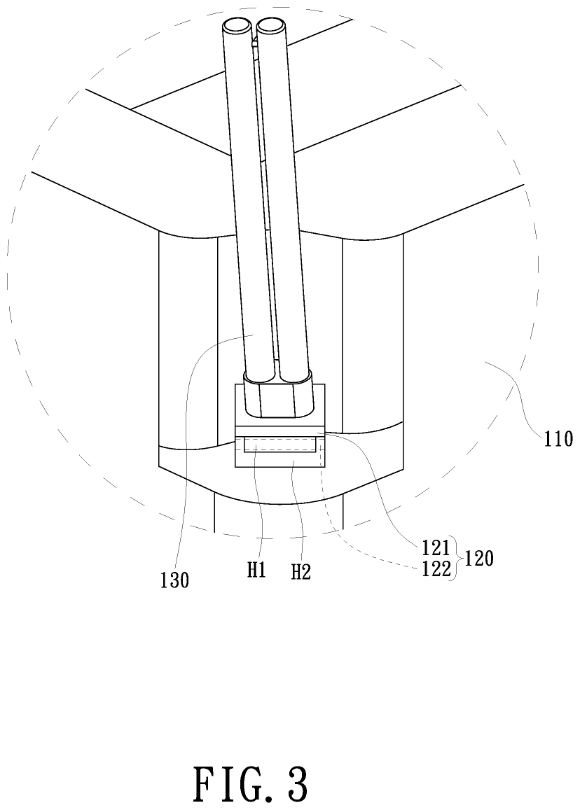

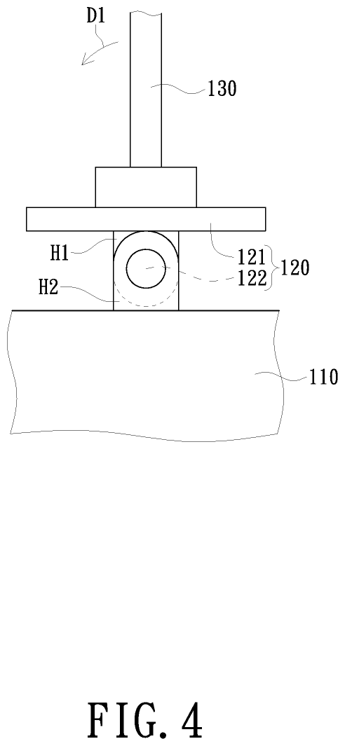

FIG. 3 is an enlarged drawing of a first disinfection light source and a first angle adjustment element in FIG. 1. FIG. 4 is an actuating diagram of the first disinfection light source and the first angle adjustment element in FIG. 1. Please refer to FIG. 3 and FIG. 4, in this embodiment, each of the first angle adjustment elements 120 can be pivoted to the base 110 and the first disinfection light source 130 to make the first disinfection light source 130 be rotate relative to the base 110. In detail, the first angle adjustment element 120 can include a first platform 121 and a first pivoting component 122. The first pivoting component 122 is pivoted to the first platform 121 and the base 110, and the first disinfection light sources 130 are respectively connected to the first platforms 121. As shown in FIG. 2, the driving element 140 is electrically connected to the first pivoting component 122, and the driving element 140 is adapted to drive the first pivoting component 122 to rotate. Please refer to FIG. 3 and FIG. 4 again, for example, the first platform 121 may have a first hinge H1 on a side facing to the base 110, and the base 110 may have a second hinge H2 corresponding to the first hinge H1. The first pivoting component 122 in this embodiment may include a cylinder, and the above cylinder can be disposed between the first hinge H1 and the second hinge H2 in a penetrating manner, thereby making the first platform 121 being rotated back and forth relative to the base 110 along a direction D1. However, in an embodiment, the first pivoting component 122 may include a sphere to provide more rotating directions for the first platform 121. The specific structure of the pivoting component is not limited in the present disclosure. By the way, the first platform 121 in this embodiment may be platy, but in other embodiments, the first platform 121 may be slightly bended, and the specific features of the first platform 121 are not limited in the present disclosure.

Please refer to FIG. 1 and FIG. 2 again, the driving element 140 in this embodiment includes, for example, a plurality of motors (not shown). Further, the plurality of motors can be electrically connected to the first pivoting components 122 respectively, so as to drive the first pivoting components 122 to rotate. In addition, the disinfection module 100 in this embodiment may further include a remote control component (not shown) corresponding to the driving element 140, and the remote control component can be communicatively connected to the driving element 140, and the remote control component can control operation of the driving element 140 via wireless signals. In another embodiment, the remote control component can be electrically connected to the driving element 140, so as to control operation of the driving element 140. By the way, the driving element 140 in this embodiment may be disposed in the base 110, but the specific position of the driving element 140 is not limited in the present disclosure.

Please continue to refer to FIG. 1, in this embodiment, the base 110 may be made from a plastic or a metal, but the present disclosure is not limited thereto. The base 110 in this embodiment can have a side surface 111, a first surface 112 and a second surface 113. The first surface 112 is opposite to the second surface 113. The side surface 111 is connected to the first surface 112 and the second surface 113, and the first surface 112 is encircled by the side surface 111. The first surface 112 has a plurality of recessed parts 1120. The recessed parts 1120 are sunken toward the second surface 113, and the recessed parts extend to the side surface 111. The first angle adjustment elements 120 and the first disinfection light sources 130 are disposed in the recessed parts 1120. Thus, the first disinfection light sources 130 can be disposed along the side surface 111 of the base 110 via the recessed parts 1120, so as to further increase the irradiation ranges of the disinfection light sources of the disinfection module 100.

Compared with prior art, the disinfection module 100 in this embodiment uses the first disinfection light sources 130 and the first angle adjustment elements 120, wherein the first angle adjustment elements 120 are connected to the first disinfection light sources 130, and the first angle adjustment elements 120 can be driven by the driving element 140 to change the irradiation angles of the first disinfection light sources 130. Therefore, the first disinfection light sources 130 of the disinfection module 100 in this embodiment have variable irradiation angles, thereby increasing the irradiation ranges of the distinction light sources 130.

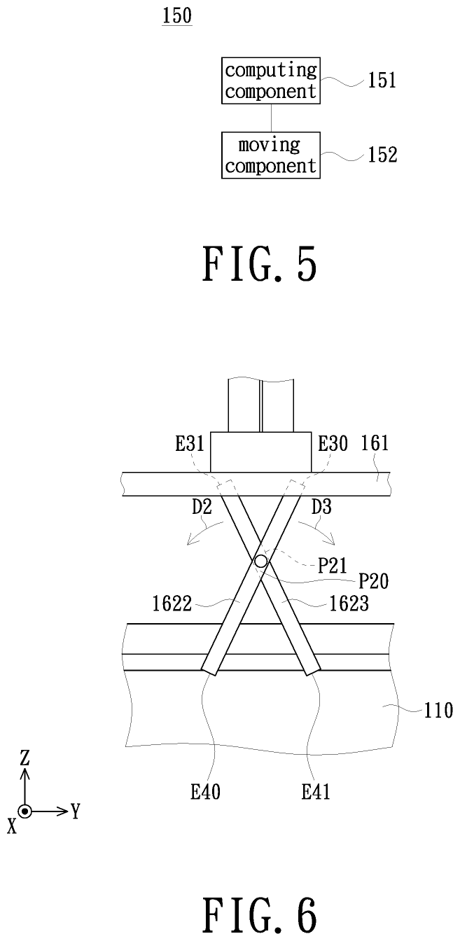

FIG. 5 is a block diagram of a moving element in FIG. 1. Please refer to FIG. 1 and FIG. 5, the disinfection module 100 (marked in FIG. 1) can further include a moving element 150, and the base 110 is disposed on the moving element 150. The moving element 150 includes a computing component 151 (shown in FIG. 5) and a moving component 152. The computing component 151 is electrically connected to the moving component 152, and the computing component 151 is adapted to drive the moving component 152 to actuate. The moving element 150 is adapted to be moved by means of actuation of the moving component 152. Thus, the disinfection module 100 can move via the moving element 150, so as to further increase the operation area of the disinfection module 100. In this embodiment, the moving element 150 includes, for example, an Autonomous Mobile Robot (AMR), but the present disclosure is not limited thereto. In addition, the computing component 151 in this embodiment may include a microcomputer, but in an embodiment, the computing component 151 may include a central processing unit. In addition, the moving component 152 in this embodiment includes, for example, a plurality of wheels, but the specific features of the moving component 152 are not limited in the present disclosure.

Please refer to FIG. 1 and FIG. 2 together again, in this embodiment, the disinfection module 100 further includes, for example, a lifting base 160 and a plurality of second disinfection light sources 170. The lifting base 160 includes a base body 161 and a connecting element 162, and the connecting element 162 is connected to the base body 161 and the base 110. The driving element 140 is further electrically connected to the connecting element 162, and the driving element 140 is further adapted to drive the base body 161 to ascend and descend relative to the base 110 via the connecting element 162. The second disinfection light sources 170 are connected to the base body 161. Thus, the quantity of the disinfection light sources of the disinfection module 100 can be further increased, and the second disinfection light sources 170 can further increase the irradiation ranges as the base body 161 ascends and descends. Features of the second disinfection light sources 170 are substantially the same as those of the first disinfection light sources 130, and related description is omitted herein.

FIG. 6 is an actuating diagram of a second rod body of the lifting base in FIG. 1, wherein perspective relations are shown in FIG. 6 and FIG. 1 in directions X, Y and Z. Please continue to refer to FIG. 1 firstly, the connecting element 162 can include first rod bodies 1620 and 1621 and second rod bodies 1622 and 1623. The two first rod bodies 1620 and 1621 are disposed across each other, and the first rod body 1620 has a first pivoting part P10, a first end E10 and a second end E20. The first end E10 is opposite to the second end E20, and the first pivoting part P10 is located between the first end E10. The first end E10 is opposite to the second end E20. The first rod body 1621 has a first pivoting part P11, a first end E11 and a second end E21. The first end E11 is opposite to the second end E21. The first pivoting part P11 is located between the first end E11 and the second end E21, and the first pivoting parts P10 and P11 are pivoted to each other. The first ends E10 and E11 are connected to the base body 161, and the second ends E20 and E21 are connected to the base 110.

Please refer to FIG. 6, similarly, the second rod bodies 1622 and 1623 are disposed across each other, wherein the second rod body 1622 has a second pivoting part P20, a third end E30 and a fourth end E40. The third end E30 is opposite to the fourth end E40, and the second pivoting part P20 is located between the third end E30. The third end E30 is opposite to the fourth end E40. The second rod body 1623 has a second pivoting part P21, a third end E31 and a fourth end E41. The third end E31 is opposite to the fourth end E41. The second pivoting part P21 is located between the third end E31 and the fourth end E41, and the second pivoting parts P20 and P21 are pivoted to each other. The third ends E30 and E31 are connected to the base body 161, and the fourth ends E40 and E41 are connected to the base 110. Please refer to FIG. 1 and FIG. 6 together, the driving element 140 (shown in FIG. 2) is further adapted to, for example, drive the second ends E20 and E21 to be close to each other and drive the fourth ends E40 and E41 to be close to each other to make the base body 161 be adapted to ascend relative to the base 110. On the other hand, the driving element 140 is further adapted to drive the second ends E20 and E21 to be away from each other and drive the fourth ends E40 and E41 to be away from each other to make the base body 161 be adapted to descend relative to the base 110.

In short, when the second ends E20 and E21 are driven to be close to each other by the driving element 140 (shown in FIG. 2), the first rod bodies 1620 and 1621 can be rotated centered on the first pivoting parts P10 and P11 pivoted to each other. Specifically, the first rod body 1620 may be rotated toward a direction D2, and the first rod body 1621 may be rotated toward a direction D3 opposite to the direction D2, so that the first ends E10 and E11 are close to each other. Similarly, when the fourth ends E40 and E41 are driven by the driving element 140 to be close to each other, the second rod bodies 1622 and 1623 can be rotated centered on the second pivoting parts P20 and P21 pivoted to each other. In detail, the second rod body 1622 may be rotated toward the direction D2, and the second rod body 1623 may be rotated towards the direction D3, so that the fourth ends E40 and E41 are close to each other. Thus, the base body 161 can be pushed toward the direction away from the base 110 by the first rod bodies 1620 and 1621 and the second rod bodies 1622 and 1623, so that the base body 161 is ascended relative to the base 110.

On the other hand, when the second ends E20 and E21 are driven to be away from each other by the driving element 140 (shown in FIG. 2), the first rod bodies 1620 and 1621 can be rotated centered on the first pivoting parts P10 and P11 pivoted to each other. Specifically, the first rod body 1620 may be rotated toward a direction D3, and the first rod body 1621 may be rotated toward the direction D2, so that the first ends E10 and E11 are away from each other. Similarly, when the fourth ends E40 and E41 are driven to be away from each other by the driving element 140, the second rod bodies 1622 and 1623 can be rotated centered on the second pivoting parts P20 and P21 pivoted to each other. In detail, the second rod body 1622 may be rotated toward the direction D3, and the second rod body 1623 may be rotated toward the direction D2, so that the fourth ends E40 and E41 are away from each other. Thus, the base body 161 can be moved toward the base 110 by the first rod bodies 1620 and 1621 and the second rod bodies 1622 and 1623, so that the base body 161 is descended relative to the base 110. By the way, in this embodiment, the second ends E20 and E21 and the fourth ends E40 and E41 can be moved by the driving element 140 via an oil pressure rod (not shown), but other embodiments are not limited thereto.

By the way, please continue to refer to FIG. 1, in this embodiment, the connecting element 162 can be connected to the first surface 112 of the base 110 and the base body 161. The first surface 112 has, for example, an accommodation groove 1121, and the base body 161 is adapted to be disposed in the accommodation groove 1121. In short, the base body 161 can be moved toward the accommodation groove 1121 in the process of descending relative to the base 110, so as to be accommodated in the accommodation groove 1121 after descending to a specific height.

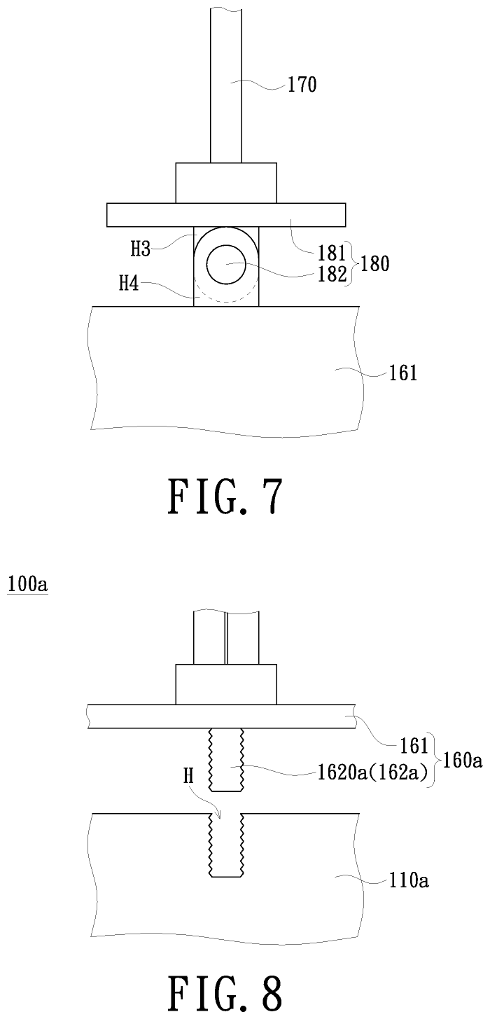

FIG. 7 is an actuating diagram of a second disinfection light source and a second angle adjustment element in FIG. 1. Please refer to FIG. 1 and FIG. 7 firstly, in this embodiment, the disinfection module 100 further includes, for example, a plurality of second angle adjustment elements 180. The second angle adjustment elements 180 are disposed on the base body 161, and the second disinfection light sources 170 are respectively connected to the second angle adjustment elements 180. Please refer to FIG. 2 and FIG. 7 together, the driving element 140 can further be electrically connected to the second angle adjustment elements 180, and the driving element 140 can further be adapted to drive the second angle adjustment elements 180 to rotate relative to the base body 161, so as to change the irradiation angles of the second disinfection light sources 170. As the function of the second angle adjustment elements 180 is similar to that of the first angle adjustment elements 120 in FIG. 1, related description is omitted herein.

Please refer to FIG. 1 and FIG. 7 together again, further, the second angle adjustment elements 180 can respectively include a second platform 181 and a second pivoting component 182. The second pivoting component 182 is pivoted to the second platform 181 and the base body 161, and the second disinfection light sources 170 are respectively connected to the second platforms 181. The driving element 140 is electrically connected to the second pivoting component 182 and is adapted to drive the second pivoting component 182 to rotate. In this embodiment, the driving element 140 may further include a plurality of motors electrically connected to the second pivoting components 182, so as to drive the second pivoting components 182 to rotate via the motors. As shown in FIG. 7, the second platform 181 may have a third hinge H3 on the side facing to the base body 161, and the third hinge H3 corresponds to, for example, a fourth hinge H4 of the base body 161. For example, the second pivoting component 182 is cylindrical, and the second pivoting component 182 is disposed on the third hinge H3 and the fourth hinge H4 in a penetrating manner, so that the second pivoting platform 181 can rotate relative to the base 110. As other features of the second pivoting components 182 are similar to those of the first pivoting components 122 in FIG. 4, related description is omitted herein. Similarly, features of the second platform 181 are similar to those of the first platform 121 in FIG. 4, so related description is omitted herein.

FIG. 8 is a schematic diagram showing separation of a connecting element of the disinfection module and a base in another embodiment of the present disclosure. The structure and advantages of the disinfection module 100a in this embodiment are similar to those in this embodiment shown in FIG. 1, and differences are merely described below. Please refer to FIG. 8, a connecting element 162a of a lifting base 160a includes, for example, a screw rod element 1620a. A base 110a can have a screw hole H. The screw rod element 1620a is disposed at the screw hole H and is connected to the base body 161. The driving element 140 (shown in FIG. 2) is adapted to drive the screw rod element 1620a to rotate relative to the base 110a at the screw hole H to make the base body 161 be adapted to ascend or descend relative to the base 110a.

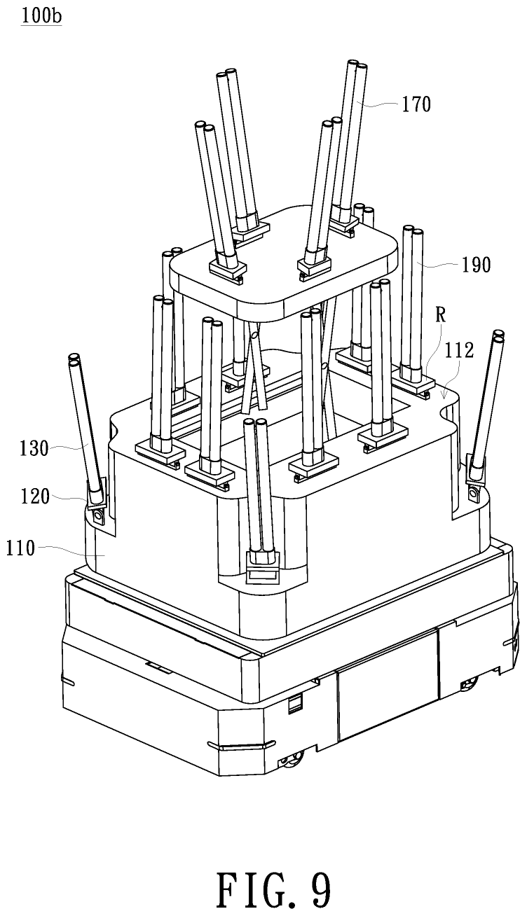

FIG. 9 is a schematic diagram of a disinfection module in another embodiment of the present disclosure. The structure and advantages of the disinfection module 100b in this embodiment are similar to those in this embodiment shown in FIG. 1, and differences are merely described below. Firstly, please refer to FIG. 1 again, it is to be understood that the quantity of the disinfection light sources can be changed according to demand in practice. For example, in this embodiment shown in FIG. 1, four first disinfection light sources 130 and four second disinfection light sources 170 are disposed, which is not limited in other embodiments. Please refer to FIG. 9, besides the first disinfection light sources 130 and the second disinfection light sources 170, the disinfection module 100b further include third disinfection light sources 190. Further, for example, the third disinfection light sources 190 are disposed on the first surface 112 of the base 110, which is not limited in the present disclosure. It is to be understood that the third disinfection light sources 190 may be connected to the base 110 via third angle adjustment parts R, so that the irradiation angles of the third disinfection light sources 190 are changed. Similarly, features of the third angle adjustment parts R may be similar to those of the first angle adjustment elements 120 in FIG. 1, so that related description is omitted herein. By the way, in this embodiment, the quantity of the third disinfection light sources 190 is not limited to that shown in FIG. 9, and more disinfection light sources can further be disposed in other positions of the base 110. In another embodiment, the shape of the base 110 may be different from that shown in FIG. 9, so that the disinfection light sources can be disposed in more varied ways. These details are not limited by the present disclosure.

In summary, the disinfection module provided by the present disclosure uses the first disinfection light sources and the first angle adjustment elements, wherein the first angle adjustment elements are connected to the first disinfection light sources and can be driven by the driving element to change the irradiation angles of the first disinfection light sources. Therefore, the first disinfection light sources of the disinfection module provided by the present disclosure have variable irradiation angles, thereby increasing the irradiation ranges of the distinction light sources.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A disinfection module, comprising:
a base;
a plurality of first angle adjustment elements respectively disposed on the base;
a plurality of first disinfection light sources respectively connected to the first angle adjustment elements; and
a driving element electrically connected to the first angle adjustment elements, and the driving element adapted to drive the first angle adjustment elements to rotate relative to the base so as to change irradiation angles of the first disinfection light sources.

2. The disinfection module according to claim 1, wherein each of the first angle adjustment elements comprises a first platform and a first pivoting component, the first pivoting component is pivoted to the first platform and the base, the first disinfection light sources are respectively connected to the first platforms, the driving element is electrically connected to the first pivoting component, and the driving element is adapted to drive the first pivoting component to rotate.

3. The disinfection module according to claim 1, wherein the base has a side surface, a first surface and a second surface, the first surface is opposite to the second surface, the side surface is connected to the first surface and the second surface, and the first surface is encircled by the side surface, the first surface has a plurality of recessed parts, the recessed parts are sunken towards the second surface, and the recessed parts extend to the side surface, the first angle adjustment elements and the first disinfection light sources are disposed in the recessed parts.

4. The disinfection module according to claim 1, further comprising a lifting base and a plurality of second disinfection light sources, wherein the lifting base comprises a base body and a connecting element, the connecting element is connected to the base body and the base, the driving element is further electrically connected to the connecting element, and the driving element is further adapted to drive the base body to ascend and descend relative to the base via the connecting element, the second disinfection light sources are connected to the base body.

5. The disinfection module according to claim 4, wherein the connecting element comprises two first rod bodies and two second rod bodies;
the two first rod bodies are disposed across each other, and the two first rod bodies each has a first pivoting part, a first end and a second end, the first end is opposite to the second end, the first pivoting part is located between the first end and the second end, and the two first pivoting parts are pivoted to each other, the two first ends are connected to the base body, and the two second ends are connected to the base;
the two second rod bodies are disposed across each other, and the two first rod bodies each has a second pivoting part, a third end and a fourth end, the third end is opposite to the fourth end, the second pivoting part is located between the third end and the fourth end, and the two second pivoting parts are pivoted to each other, the two third ends are connected to the base body, and the two fourth ends are connected to the base; and
the driving element is further adapted to drive the two second ends to be close to each other and drive the two fourth ends to be close to each other to make the base body be adapted to ascend relative to the base, and the driving element is further adapted to drive the two second ends to be away from each other and drive the two fourth ends to be away from each other to make the base body be adapted to descend relative to the base.

6. The disinfection module according to claim 4, wherein the connecting element comprises a screw rod element; the base has a screw hole, the screw rod element is disposed at the screw hole, and the screw rod element is connected to the base body, the driving element is adapted to drive the screw rod element to rotate relative to the base at the screw hole to make the base body be adapted to ascend or descend relative to the base.

7. The disinfection module according to claim 4, further comprising a plurality of second angle adjustment elements, wherein the second angle adjustment elements are disposed on the base body, and the second disinfection light sources are respectively connected to the second angle adjustment elements. the driving element is further electrically connected to the second angle adjustment elements, and the driving element is further adapted to drive the second angle adjustment elements to rotate relative to the base body so as to change the irradiation angles of the second disinfection light sources.

8. The disinfection module according to claim 4, wherein the second angle adjustment elements each comprises a second platform and a second pivoting component, the second pivoting component is pivoted to the second platform and the base body, and the second disinfection light sources are respectively connected to the second platforms, the driving element is electrically connected to the second pivoting component, and the driving element is adapted to drive the second pivoting component to rotate.

9. The disinfection module according to claim 4, wherein the base has a first surface, the first surface faces to the base body, and the connecting element is connected to the first surface and the base body, the first surface has an accommodation groove, and the base body is adapted to be disposed in the accommodation groove.

10. The disinfection module according to claim 1, further comprising a moving element, wherein the base is disposed on the moving element, the moving element comprising a computing component and a moving component, the computing component is electrically connected to the moving component, and the computing component is adapted to drive the moving component to actuate, the moving element is adapted to be moved by means of actuation of the moving component.
